(19) United States
(12) Patent Application Publication
(10) Pub. No.: US 2005/0026933 A1
(43) Pub. Date: Feb. 3, 2005

(54) Use of a combination of an epidermal growth factor receptor kinase inhibitor and cytotoxic agents for treatment and inhibition of cancer

(75) Inventors: Lee Greenberger, Montclair, NJ (US); Carolyn Mary Discafani-Marro, Cortlandt Manor, NY (US); Philip Frost, Morris Township, NJ (US)

Correspondence Address: WYETH;PATENT LAW GROUP, 5 GIRALDA FARMS, MADISON, NJ, 07940, US

(73) Assignee: Wyeth Holdings Corporation, Madison, NJ (US)

(21) Appl. No.: 10/900,655

(22) Filed: Jul. 28, 2004

Related U.S. Application Data
(60) Provisional application No. 60/492,132, filed on Aug. 1, 2003.

(51) Int. Cl. A61K 31/506; A61K 31/4709

(52) U.S. Cl. 514/256; 514/314

(57) ABSTRACT

This invention discloses a method of treating or inhibiting cancer in a mammal in need thereof which comprises administering to said mammal an effective amount of a cytotoxic agent and an EGFR kinase inhibitor.

This application claims priority from copending provisional application Ser. No. 60/492,132, filed Aug. 1, 2003, the entire disclosure of which is hereby incorporated by reference.

### BACKGROUND OF THE INVENTION

This invention relates to the use of a combination of a cytotoxic agent and an epidermal growth factor receptor (EGFR) kinase inhibitor in the treatment and inhibition of cancer.

Protein tyrosine kinases are a class of enzymes that catalyze the transfer of a phosphate group from ATP or GTP to tyrosine residue located on protein substrates. Protein tyrosine kinases clearly play a role in normal cell growth. Many of the growth factor receptor proteins function as tyrosine kinases and it is by this process that they effect signaling. The interaction of growth factors with these receptors is a necessary event in normal regulation of cell growth. However, under certain conditions, as a result of either mutation or over expression, these receptors can become deregulated; the result of which is uncontrolled cell proliferation which can lead to tumor growth and ultimately to the disease known as cancer [Wilks A. F., Adv. Cancer Res., 60, 43 (1993) and Parsons, J. T.; Parsons, S. J., Important Advances in Oncology, DeVita V. T. Ed., J. B. Lippincott Co., Phila., 3 (1993)]. Among the growth factor receptor kinases and their proto-oncogenes that have been identified and which are targets of the compounds of this invention are the epidermal growth factor receptor kinase (EGFR kinase, the protein product of the erbB oncogene), and the product produced by the erbB-2 (also referred to as the neu or HER2) oncogene. Since the phosphorylation event is a necessary signal for cell division to occur and since overexpressed or mutated kinases have been associated with cancer, an inhibitor of this event, a protein tyrosine kinase inhibitor, will have therapeutic value for the treatment of cancer and other diseases characterized by uncontrolled or abnormal cell growth. For example, over expression of the receptor kinase product of the erbB-2 oncogene has been associated with human breast and ovarian cancers [Slamon, D. J., et. al., Science, 244, 707 (1989) and Science, 235, 1146 (1987)]. Deregulation of EGF-R kinase has been associated with epidermoid tumors [Reiss, M., et. al., Cancer Res., 51, 6254 (1991)], breast tumors [Macias, A., et. al., Anticancer Res., 7, 459 (1987)], and tumors involving other major organs [Gullick, W. J., Brit. Med. Bull., 47, 87 (1991)]. Because of the importance of the role played by deregulated receptor kinases in the pathogenesis of cancer, many recent studies have dealt with the development of specific PTK inhibitors as potential anti-cancer therapeutic agents [some recent reviews: Burke. T. R., Drugs Future, 17, 119 (1992) and Chang, C. J.; Geahlen, R. L., J. Nat. Prod, 55, 1529 (1992)].

An EGFR kinase inhibitor of interest is (4-dimethylamino-but-2-enoic acid [4-(3-chloro-4-fluoro-phenylamino)-3-cyano-7-ethoxy-quinolin-6-yl]-amide (EKB-569. While it is important that EKB-569 works as a single anti-cancer agent, it is possible that tyrosine kinase inhibitors may be most effective when given in combination with established chemotherapeutic agents. The studies in this report were designed to determine if EKB-569 in combination with conventional chemotherapeutic agents (cytotoxic agents) provide better tumor growth inhibition than when either drug is administered alone.

### BRIEF SUMMARY OF THE INVENTION

The present invention relates to a method of treating or inhibiting cancer in a mammal in need thereof that comprises administering to said mammal an effective amount of a cytotoxic agent and an EGFR kinase inhibitor.

The following experimental details are set forth to aid in an understanding of the invention, and are not intended, and should not be construed, to limit in any way the invention set forth in the claims that follow thereafter.

### DETAILED DESCRIPTION OF THE INVENTION

This invention provides a method of treating or inhibiting cancer in a mammal in need thereof, which comprises administering to said mammal a cytotoxic agent and an EGFR kinase inhibitor.

For the purpose of defining the scope of this invention, an EGFR kinase inhibitor is defined as a molecule that inhibits the kinase domain of the EGFR. It is preferred that the EGFR kinase inhibitor irreversibly inhibits EGFR kinase, typically by possessing a reactive moiety (such as a Michael acceptor) that can form a covalent bond with EGFR.

For purposes of this invention the EGFR kinase inhibitor includes, the following:

Quinazolines of Formula 1, which are disclosed in U.S. Pat. No. 6,384,051 B1. These compounds can be prepared according to the methodology described in U.S. Pat. No. 6,384,051 B1, which is hereby incorporated by reference. The structure of the EGFR kinase inhibitors of Formula 1 are as follows: 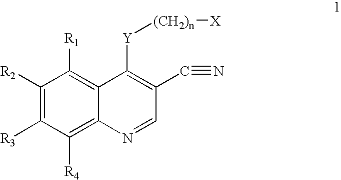
wherein:

X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;

n is 0-1;

Y is —NH—, —O—, —S—, or —NR—;

R is alkyl of 1-6 carbon atoms;

R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino,

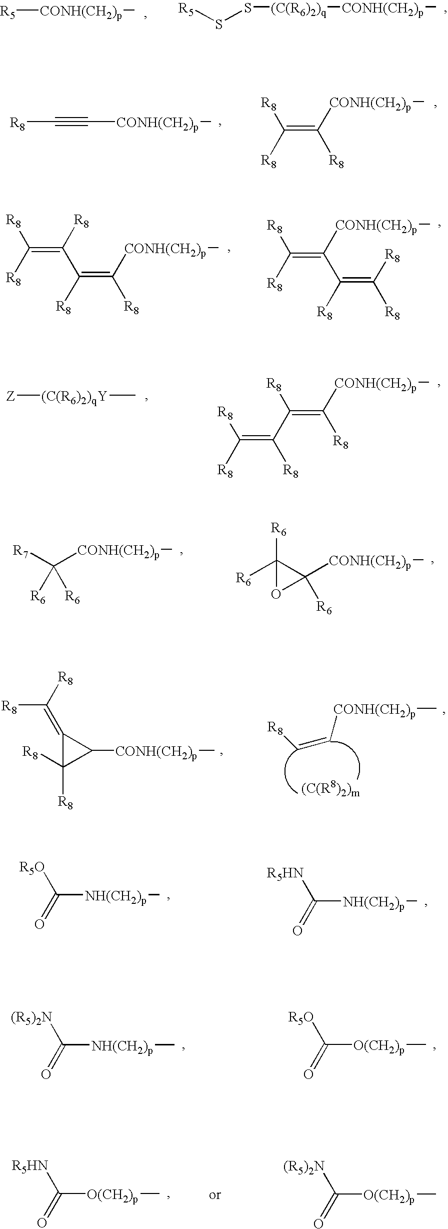

R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;

R₆ is hydrogen, alkyl of 1-6 carbon atoms, or alkenyl of 2-6 carbon atoms;

R₇ is chloro or bromo;

R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;

Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;

m=1-4, q=1-3, and p=0-3;

any of the substituents R₁, R₂, R₃, or R₄ that are located on contiguous carbon atoms can together be the divalent radical —O—C(R₈)₂—O—;

or a pharmaceutically acceptable salt thereof with the proviso that when Y is —NH—, R₁, R₂, R₃, and R₄ are hydrogen, and n is 0, X is not 2-methylphenyl.

With respect to the cyanoquilines of Formula 1, the pharmaceutically acceptable salts are those derived from such organic and inorganic acids as: acetic, lactic, citric, tartaric, succinic, maleic, malonic, gluconic, hydrochloric, hydrobromic, phosphoric, nitric, sulfuric, methanesulfonic, and similarly known acceptable acids.

The alkyl portion of the alkyl, alkoxy, alkanoyloxy, alkoxymethyl, alkanoyloxymethyl, alkylsulphinyl, alkylsulphonyl, alkylsulfonamido, carboalkoxy, carboalkyl, alkanoylamino aminoalkyl, alkylaminoalkyl, N,N-dicycloalkylaminoalkyl, hydroxyalkyl, and alkoxyalkyl substituents include both straight chain as well as branched carbon chains. The cycloalkyl portions of N-cycloalkyl-N-alkylaminoalkyl and N,N-dicycloalkylaminoalkyl substituents include both simple carbocycles as well as carbocycles containing alkyl substituents. The alkenyl portion of the alkenyl, alkenoyloxymethyl, alkenyloxy, alkenylsulfonamido, substituents include both straight chain as well as branched carbon chains and one or more sites of unsaturation. The alkynyl portion of the alkynyl, alkynoyloxymethyl, alkynylsulfonamido, alkynyloxy, substituents include both straight chain as well as branched carbon chains and one or more sites of unsaturation. Carboxy is defined as a —CO₂H radical. Carboalkoxy of 2-7 carbon atoms is defined as a —CO₂R″ radical, where R″ is an alkyl radical of 1-6 carbon atoms. Carboalkyl is defined as a —COR″ radical, where R″ is an alkyl radical of 1-6 carbon atoms. Alkanoyloxy is defined as a —OCOR″ radical, where R″ is an alkyl radical of 1-6 carbon atoms. Alkanoyloxymethyl is defined as R″CO₂CH₂— radical, where R″ is an alkyl radical of 1-6 carbon atoms. Alkoxymethyl is defined as R″OCH₂— radical, where R″ is an alkyl radical of 1-6 carbon atoms. Alkylsulphinyl is defined as R″SO— radical, where R″ is an alkyl radical of 1-6 carbon atoms. Alkylsulphonyl is defined as R″SO₂— radical, where R″ is an alkyl radical of 1-6 carbon atoms. Alkylsulfonamido, alkenylsulfonamido, alkynylsulfonamido are defined as R″SO₂NH— radical, where R″ is an alkyl radical of 2-6 carbon atoms, an alkenyl radical of 2-6 carbon atoms, or an alkynyl radical of 2-6 carbon atoms, respectively. When X is substituted, it is preferred that it is mono-, di- , or tri-substituted, with monosubstituted being most preferred. It is preferred that of the substituents R₁, R₂, R₃, and R₄, at least one is hydrogen and it is most preferred that two or three be hydrogen. An azacycloalkyl-N-alkyl substituent refers to a monocyclic heterocycle that contains a nitrogen atom on which is substituted a straight or branched chain alkyl radical. A morpholino-N-alkyl substituent is a morpholine ring substituted on the nitrogen atom with a straight or branch chain alkyl radical. A piperidino-N-alkyl substituent is a piperidine ring substituted on one of the nitrogen atoms with a straight or branch chain alkyl radical. A N-alkyl-piperidino-N-alkyl substituent is a piperidine ring substituted on one of the nitrogen atoms with a straight or branched chain alkyl group and on the other nitrogen atom with a straight or branch chain alkyl radical.

The term alkyl includes both straight and branched chain alkyl moieties, preferably of 1-6 carbon atoms. The term alkenyl includes both straight and branched alkenyl moieties of 2-6 carbon atoms containing at least one double bond. Such alkenyl moieties may exist in the E or Z conformations; the compounds of this invention include both conformations. The term alkynyl includes both straight chain and branched alkynyl moieties containing 2-6 carbon atoms containing at least one triple bond. The term cycloalkyl refers to an alicyclic hydrocarbon group having 3-7 carbon atoms.

The term halogen is defined as Cl, Br, F, and I.

Alkoxy, alkylthio, alkoxyalkyl, alkylthioalkyl, alkoxyalkyloxy and alkylthioalkyloxy are moieties wherein the alkyl chain is 1-6 carbon atoms (straight or branched).

The term alkylamino refers to moieties with one or two alkyl groups wherein the alkyl chain is 1-6 carbons and the groups may be the same or different. The alkyl groups (the same or different) bonded to the nitrogen atom which is attached to an alkyl group of 1-3 carbon atoms.

The compounds of Formula 1 may contain an asymmetric carbon; in such cases, the compounds of Formula 1 cover the racemate and the individual R and S entantiomers, and in the case were more than one asymmetric carbon exists, the individual diasteromers, their racemates and individual entantiomers.

For purposes of this invention an EGFR kinase inhibitor of interest having a structure of formula 1 includes (4-dimethylamino-but-2-enoic acid [4-(3-chloro-4-fluoro-phenylamino)-3-cyano-7-ethoxy-quinolin-6-yl]-amide) (“EKB-569”).

The chemical structures of cytotoxic agents vary. Certain cytotoxic agents include, but are not limited to; capecitabine, paclitaxel, 5-Fluorouracil (5-FU), FOLFIRI, FOLFOX4 (Fluorouracil/Leucovorin/Oxaliplatin) and cisplatin. The cytotoxic agents of this invention are either commercially available or can be prepared by standard literature procedures.

For purposes of this invention cancer includes colorectal and pancreatic cancer.

Multiple experiments were performed in order to determine if EKB-569 given in combination with conventional cytotoxic agents was capable of treating or inhibiting in vivo tumor growth significantly better than if either drug was given alone. In all of the experiments, athymic nu/nu female mice (Charles River Laboratories) were injected SC (subcutaneously) with either 7×10⁶ or 1×10⁷ LoVo colon carcinoma cells or 5×10⁶ GEO colon carcinoma cells. When tumors attained a mass of between 80 and 120 mg (Day 0), animals were randomized into treatment groups each containing between 5 and 20 animals, (dependent upon the experiment). Mice were treated orally (PO) with EKB-569 or vehicle control for 15 to 20 days, depending upon the experiment. EKB-569 was formulated in 0.5% Methocel, 0.4% Tween 80. Cytotoxic agents, (paclitaxel, 5-FU and cisplatin), were given either by parenteral (IP) or intravenous (IV) administration on either days 1, 5 and 9 or on days 1, 5, 9 and 13, depending upon the experiment. Tumor mass ([Length×Width²]/2) was determined every seven days post staging for up to 35 days. The relative tumor growth, (Mean tumor mass on day measured divided by the mean tumor mass on day zero), and the percent Tumor/Control, (% T/C), was then calculated for each treatment group for as long as the control group remained. The % T/C is defined as the Mean Relative Tumor Growth of the Treated Group divided by the Mean Relative Tumor Growth of Vehicle Control Group multiplied by 100. The data was analyzed via Student's one-tailed t-test. A p-value≦0.05 indicates a statistically significant reduction in relative tumor growth of treated group compared with the vehicle control group or drug treated group.

The activity of EKB-569 in combination with paclitaxel was assessed using the human colon carcinoma lines LoVo and GEO. In the LoVo experiment, 20 mg/kg EKB-569 was administered PO for 20 consecutive days. Twenty mg/kg paclitaxel prepared in 2% cremophor el and 2% ethanol was administered IV on days 1, 5, 9 and 13. In this study, EKB-569 administered alone resulted in between 25 and 59% tumor growth inhibition. Treatment with paclitaxel alone resulted in 41 to 74% growth inhibition; the effects of paclitaxel diminished after dosing was terminated (FIG. 1). The 2 drugs administered in combination resulted in approximately 80% tumor growth inhibition from day 14 until the end of the experiment on day 35. Statistical analysis via Student's t-Test revealed that the combination therapy was statistically superior compared with paclitaxel treatment alone at 3 out of 5 time points (p≦0.05).

In the experiment of Table 1 (GEO), 80 mg/kg EKB-569 was administered PO for 15 consecutive days while 25 mg/kg paclitaxel was administered IV on days 1, 5, 9 and 13 (Table 1). The results obtained in this study were identical to that in the LoVo study except that there was up to 85% tumor growth inhibition seen in the group receiving combination therapy. This inhibition was significantly different than either compound administered alone at virtually all time points.

The activity of EKB-569 in combination with 5-FU was assessed in LoVo and GEO xenografts. In the LoVo experiment, 20 mg/kg EKB-569 was administered for 20 consecutive days while 40 mg/kg 5-FU was administered IP on days 1, 5, 9 and 13. In the GEO experiment, 80 mg/kg EKB-569 was administered PO for 15 consecutive days while 40 mg/kg 5-FU was administered IP on days 1, 5, 9 and 13. In both the LoVo (Table 2) and the GEO experiments (Table 3), the combination of EKB-569 and 5-FU was capable of inhibiting tumor growth significantly better than in the groups that received either 5-FU or EKB-569 alone at one or more time points examined (p≦.0.05). At all time points, tumor size was smaller in the combination group compared with the single agent groups in these experiments.

The activity of EKB-569 in combination with cisplatin was assessed in LoVo and GEO xenografts. In the LoVo study, 20 mg/kg EKB-569 was administered PO for 20 consecutive days while 3 mg/kg cisplatin was administered IP on days 1, 5 and 9. In the GEO experiment, 80 mg/kg EKB-569 was administered PO for 15 consecutive days while 3 mg/kg cisplatin was administered I P on days 1, 5, 9 and 13. In both these experiments, combination therapy gave statistically significant, (p≦0.05), tumor growth inhibition than either drug alone at 3 out of 4 time points examined. Greater than 70% inhibition was seen in the combination group of both studies where EKB-569 or cisplatin gave no more than 50% inhibition in either experiment.

In each experiment, all groups receiving the combination therapy showed an increase in the percent tumor growth inhibition compared with each drug alone. In the 5-FU experiments, the combination group had between 12 and 42% increase in growth inhibition compared with the animals receiving EKB-569 alone and between 11 and 37% compared with animals receiving 5-FU. More significant inhibition was seen in the paclitaxel experiments where the combination group had between 20 and 56% increase in growth inhibition compared with the animals receiving EKB-569 alone and between 11 and 40% compared with animals receiving paclitaxel. Cisplatin showed the greatest difference in tumor growth inhibition, 18-53% compared with EKB-569 alone and 16 to 79% compared with cisplatin alone. Statistically, when the cisplatin/EKB-569 or paclitaxel/EKB-569 combination groups were compared with each drug alone, inhibitory effects at the majority of the time points were statistically superior to that of each individual drug, (p≦0.05).

TABLE 1
Effect of EKB-569 in combination with paclitaxel
in the human colon carcinoma

| | | | | p-value | p-value |
|---|---|---|---|---|---|
| | | | | vs | vs |
| Therapy | Day | % T/C^{a} | RTG^{b} | EKB-569^{c} | Paclitaxel^{d} |
| EKB-569 (80 mg/kg PO) | 6 | 51 | 1.78 | | |
| Paclitaxel (25 mg/kg IV) | | 46 | 1.63 | | |
| Combination Therapy | | 31 | 1.08 | <0.01 | <0.01 |
| EKB-569 (80 mg/kg PO) | 15 | 53 | 3.33 | | |
| Paclitaxel (25 mg/kg IV) | | 43 | 2.69 | | |
| Combination Therapy | | 15 | 0.91 | <0.01 | <0.01 |
| EKB-569 (80 mg/kg PO) | 21 | e | 5.19 | | |
| Paclitaxel (25 mg/kg IV) | | e | 2.24 | | |
| Combination Therapy | | e | 1.64 | <0.01 | 0.03 |
| EKB-569 (80 mg/kg PO) | 28 | e | 5.78 | | |
| Paclitaxel (25 mg/kg IV) | | e | 2.70 | | |
| Combination Therapy | | e | 2.10 | <0.01 | 0.14 |
| ^{a}Groups of 10 to 20 female nu/nu mice bearing staged tumors were administered either vehicle alone, 80 mg/kg EKB-569 PO on days 1 through 15, 25 mg/kg paclitaxel IV on days 1, 5, 9 and 13 or a combination of the 2 drugs. Data are presented as % Tumor/control T/C. The % T/C is defined as the Mean Relative Tumor Growth of the Treated Group divided by the Mean Relative Tumor Growth of the Vehicle control Group multiplied by 100. | | | | | |
| #Relative tumor growth is defined as the mean tumor mass on day measured divided by the mean tumor mass on day zero. | | | | | |

^{b}Relative Tumor Growth is defined as the mean tumor mass on a given day divided by the mean tumor mass on day zero.
^{c}P-values for combination therapy verses EKB-569 determined by Student's t-Test.
^{d}P-values for combination therapy verses paclitaxel determined by Student's t-Test.
^{e}Vehicle control animals sacrificed on day 15 due to tumor size.

TABLE 2
Effect of EKB-569 in combination with 5-FU
in the human colon carcinoma LoVo

| | | | p-value | p-value |
|---|---|---|---|---|
| Therapy | Day | % T/C^{a} | to EKB-569^{b} | to 5-FU^{c} |
| EKB-569 (20 mg/kg PO) | 7 | 84 | | |
| 5-FU (40 mg/kg IP) | | 59 | | |
| Combination Therapy | | 42 | <0.01 | 0.04 |
| EKB-569 (20 mg/kg PO) | 14 | 67 | | |
| 5-FU (40 mg/kg IP) | | 63 | | |
| Combination Therapy | | 40 | 0.02 | 0.12 |
| EKB-569 (20 mg/kg PO) | 21 | 77 | | |
| 5-FU (40 mg/kg IP) | | 85 | | |
| Combination Therapy | | 48 | 0.01 | 0.01 |
| EKB-569 (20 mg/kg PO) | 29 | 95 | | |
| 5-FU (40 mg/kg IP) | | 77 | | |
| Combination Therapy | | 53 | 0.01 | 0.08 |

^{a}Groups of 5 to 10 female nu/nu mice bearing staged tumors were administered either vehicle alone, 20 mg/kg EKB-569 PO on days 1 through 20, 40 mg/kg 5-FU IP on days 1, 5, 9 and 13 or a combination of the 2 drugs. Data are presented as % T/C.
^{b}P-values for combination therapy verses EKB-569 determined by Student's t-Test.
^{c}P-values for combination therapy verses 5-FU determined by Student's t-Test.

TABLE 3
Effect of EKB-569 in combination with 5-FU
in the human colon carcinoma GEO

| | | | p-value | p-value |
|---|---|---|---|---|
| Therapy | Day | % T/C^{a} | to EKB-569^{b} | to 5-FU^{c} |
| EKB-569 (80 mg/kg PO) | 8 | 55 | | |
| 5-FU (40 mg/kg IP) | | 58 | | |
| Combination Therapy | | 43 | 0.18 | 0.03 |
| EKB-569 (80 mg/kg PO) | 14 | 54 | | |
| 5-FU (40 mg/kg IP) | | 47 | | |
| Combination Therapy | | 26 | <0.01 | <0.01 |
| EKB-569 (80 mg/kg PO) | 21 | 81 | | |
| 5-FU (40 mg/kg IP) | | 50 | | |
| Combination Therapy | | 41 | 0.01 | 0.23 |
| EKB-569 (80 mg/kg PO) | 28 | 95 | | |
| 5-FU (40 mg/kg IP) | | 60 | | |
| Combination Therapy | | 54 | 0.11 | 0.31 |
| ^{a}Groups of 10 to 15 female nu/nu mice bearing staged tumors were administered either vehicle alone, 80 mg/kg EKB-569 PO on days 1 through 15, 40 mg/kg 5-FU IP on days 1, 5, 9 and 13 or a combination of the 2 drugs. Data are presented as % T/C. The % T/C is defined as the Mean Relative Tumor Growth of the Treated Group divided by the Mean Relative Tumor Growth of the | | | | |
| #Vehicle Control Group multiplied by 100. Relative tumor growth is defined as the mean tumor mass on day measured divided by the mean tumor mass on day zero. | | | | |

^{b}P-values for combination therapy verses EKB-569 determined by Student's t-Test.
^{c}P-values for combination therapy verses 5-FU determined by Student's t-Test.

TABLE 4
Weeks With Clinical Benefit:^{a}
Number of Evaluable Patients^{b,c}

| | | 25 mg EKB-569 | 50 mg EKB-569 | 50 mg EKB-569 | 75 mg EKB-569 |
|---|---|---|---|---|---|
| | Total | 750 mg/m2 CAPE | 750 mg/m2 CAPE | 1000 mg/m2 CAPE | 1000 mg/m2 CAPE |
| Weeks^{d} | N = 29 | n = 5 | n = 6 | n = 16 | n = 2 |
| ≧6 and <12 | 4 | 0 | 1 | 3 | 0 |
| ≧12 and <18 | 4 | 1 | 1 | 2 | 0 |
| ≧18 and <24 | 0 | 0 | 0 | 0 | 0 |
| ≧24 and <30 | 3 | 1 | 1 | 1 | 0 |
| ≧30 and <36 | 1 | 0 | 0 | 1 | 0 |

^{a}Clinical benefit = CR + PR + SD.
^{b}Preliminary data from Nov. 04, 2003.
^{c}Evaluable patients completed 2 cycles and had at least 1 follow-up disease assessment. Patients who discontinued before completing 2 cycles because of PD or AEs were included based on clinical review.
^{d}Tumor assessment done on alternate 21-day cycles (every 6 weeks at weeks 2-3 of even cycles). Weeks measured from the first treatment day to the day of last assessment of SD, PR, or CR.

When patients with advanced colorectal cancer were treated with the combination of EKB-569 and capecitabine:

The MTD was 50 mg EKB-569, 1000 mg/m2 capecitabine based on DLTs at 75 mg EKB-569, 1000 mg/m2 capecitabine of grade 3 diarrhea (1 patient) and grade 2 diarrhea and grade 2 rash (1 patient);

The most frequently occurring EKB-569-related treatment-emergent adverse events, all grades, were diarrhea (75%), nausea (56%), asthenia (53%), rash (45%), and anorexia (36%);

No grade 4 EKB-569-related treatment-emergent adverse events occurred;

One patient had a partial response for an objective tumor response rate of 3%. The clinical benefit rate (CR+PR+SD) was 45%; and

EKB-569 in combination with capecitabine was generally well tolerated and had antitumor activity.

TABLE 5
Months with Clinical Benefit:^{a} Number of Evaluable Patients^{b,c}

| | | 10 mg EKB = 569 | 25 mg EKB-569 | 50 mg EKB-569 | 75 mg EKB-569 | 35 EKB-569 | 50 mg EKB-569 |
|---|---|---|---|---|---|---|---|
| | All | FOLFIRI | FOLFIRI | FOLFIRI | FOLFIRI | FOLFIRI | MOD FOL |
| Months^{d} | n = 39 | n = 4 | n = 19 | n = 5 | n = 3 | n = 8 | n = 2 |
| >2 and <4 | 11 | 0 | 5 | 1 | 0 | 3 | 2 |
| ≧4 and <6 | 7 | 1 | 2 | 0 | 1 | 3 | 0 |
| ≧6 and <8 | 3 | 0 | 2 | 1 | 0 | 0 | 0 |
| ≧8 and <10 | 6 | 2 | 2 | 1 | 1 | 0 | 0 |
| >10 and <12 | 3 | 0 | 2 | 0 | 1 | 0 | 0 |
| ≧12 and <14 | 2 | 0 | 1 | 1 | 0 | 0 | 0 |
| ≧14 and <16 | 1 | 1 | 0 | 0 | 0 | 0 | 0 |

^{a}Clinical benefit = CR + PR + SD.
^{b}Preliminary data of Nov. 04, 2003.
^{c}Evaluable patients completed 2 cycles and had at least 1 follow-up disease assessment. Patients who discontinued before completing 2 cycles due to PD or AEs were included based on clinical review.
^{d}Tumor assessment done every 2 months at weeks 6-8 of even months. Months measured from first treatment day to day of last assessment of SD, PR, or CR.

When patients with advanced colorectal cancer were treated with the combination of EKB-569 and FOLFIRI:

The MTD was 25 mg EKB-569, FOLFIRI based on:

DLTs of grade 3 asthenia (1 patient, 50 mg EKB-569, FOLFIRI) and grade 3 diarrhea (2 patients, 75 mg EKB-569, FOLFIRI);

Development of diarrhea in all patients who received 50 mg EKB-569, FOLFIRI and 75 mg EKB-569, FOLFIRI;

The most frequently occurring EKB-569-related treatment-emergent adverse events, all grades, were diarrhea (75%), asthenia (51%), nausea (42%), and rash (33%);

No grade 4 EKB-569-related treatment-emergent adverse events occurred;

Three patients had complete responses and 12 had partial responses, for an objective response rate of 38%. The clinical benefit rate (CR+PR+SD) was 85%; and

EKB-569 in combination with FOLFIRI was generally well tolerated, and the combination showed clear evidence of antitumor activity.

TABLE 6
EKB-569, FOLFOX4: Best Tumor Responses

| | | 25 mg EKB-569 | 35 mg EKB-569 |
|---|---|---|---|
| | All | FOLFOX4 | FOLFOX4 |
| Best Response^{a,b} | n = 25 | n = 19 | n = 6 |
| Complete response | 0 | 0 | 0 |
| Partial response | 12 (48) | 10 (53) | 2 (33) |
| Stable disease | 12 (48) | 9 (47) | 3 (50) |
| Progressive disease | 1 (4) | 0 | 1 (17) |

^{a}Defined according to RECIST guidelines.
^{b}Preliminary data from May 03, 04 of number of evaluable patients who completed 2 cycles and had at least 1 follow-up assessment. Patients who discontinued before completing 2 cycles because of PD were included.

EKB-569 plus FOLFIRI/FOLFOX4 combinations were generally well tolerated and showed antitumor activity in patients with advanced colorectal cancer.

An ascending-dose study of the safety, tolerability, and pharmacokinetics of EKB-569 in patients with tumor types known to overexpress epidermal growth factor receptors was performed. The following cytotoxic agents were tested in combination with EKB-569 for colorectal or pancreatic cancer: gemcitabine (pancreas); 5-FU/LV/irinotecan (colorectal); capecitabine (colorectal); and 5-FU/LV/oxaliplatin (colorectal). Of the five patients treated with a combination of EKB-569 and gemcitabine, 2 had stable disease for longer than 10 months.

This invention provides to a mammal, a pharmaceutical composition that comprises a compound of formula 1 in combination or association with a pharmaceutically acceptable carrier. In a preferred embodiment the compound of formula 1 is EKB-569. The compound of this invention may be administered alone or in combination with cytotoxic agents.

Administering the pharmaceutical composition to the mammal requires delivery to the mammal in a form such as a tablet or a capsule. Delivery may occur hourly, daily, weekly, or monthly. The effective amount of the pharmaceutical composition provided to the mammal can be determined by one of skill in the art and will depend on variables such as size and age. One of skill in the art could routinely perform empirical activity tests to determine the effective amount.

1. A method of treating or inhibiting cancer in a mammal in need thereof which comprises administering to said mammal an effective amount of a cytotoxic agent and an EGFR kinase inhibitor.

2. The method according to claim 1, wherein the cytotoxic agent is selected from the group consisting of capecitabine, paclitaxel, 5-FU and cisplatin.

3. The method according to claim 1, wherein the EGFR kinase inhibitor irreversibly inhibits EGFR kinase.

4. The method according to claim 1, wherein the EGFR kinase inhibitor is a compound of formula 1, having the structure: 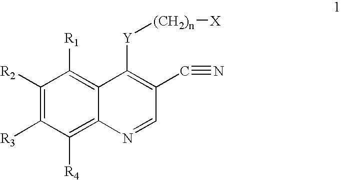
wherein:
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring may be optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
n is 0-1;
Y is —NH—, —O—, —S—, or —NR—;
R is alkyl of 1-6 carbon atoms;
R₁, R₂, R₃, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzyl, amino, hydroxyamino, alkoxyamino of 1-4 carbon atoms, alkylamino of 1-6 carbon atoms, dialkylamino of 2 to 12 carbon atoms, aminoalkyl of 1-4 carbon atoms, N-alkylaminoalkyl of 2-7 carbon atoms, N,N-dialkylaminoalkyl of 3-14 carbon atoms, phenylamino, benzylamino, 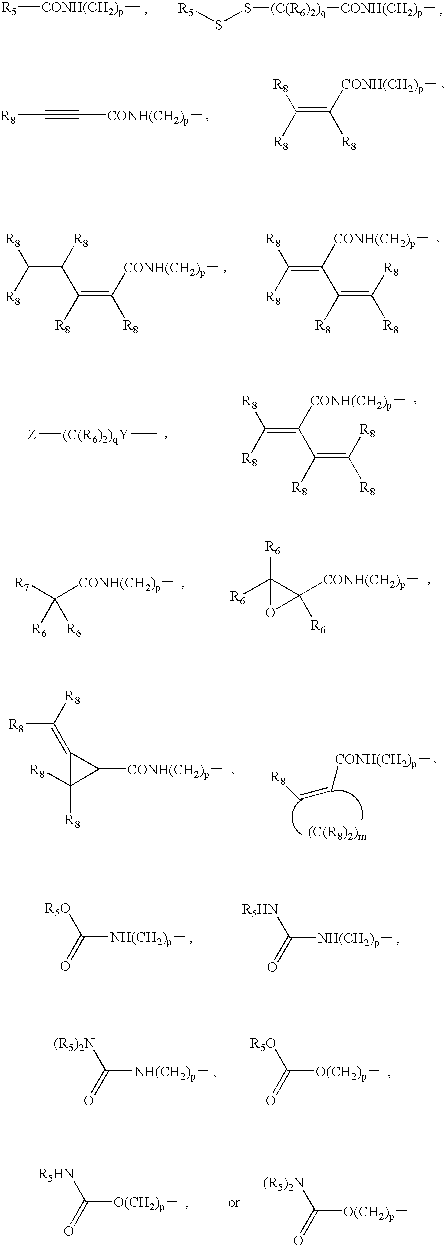
R₅ is alkyl of 1-6 carbon atoms, alkyl optionally substituted with one or more halogen atoms, phenyl, or phenyl optionally substituted with one or more halogen, alkoxy of 1-6 carbon atoms, trifluoromethyl, amino, nitro, cyano, or alkyl of 1-6 carbon atoms groups;
R₆ is hydrogen, alkyl of 1-6 carbon atoms, or alkenyl of 2-6 carbon atoms;
R₇ is chloro or bromo;
R₈ is hydrogen, alkyl of 1-6 carbon atoms, aminoalkyl of 1-6 cabon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 15 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms, azacycloalkyl-N-alkyl of 3-11 carbon atoms, hydroxyalkyl of 1-6 carbon atoms, alkoxyalkyl of 2-8 carbon atoms, carboxy, carboalkoxy of 1-6 carbon atoms, phenyl, carboalkyl of 2-7 carbon atoms, chloro, fluoro, or bromo;
Z is amino, hydroxy, alkoxy of 1-6 carbon atoms, alkylamino wherein the alkyl moiety is of 1-6 carbon atoms, dialkylamino wherein each of the alkyl moieties is of 1-6 carbon atoms, morpholino, piperazino, N-alkylpiperazino wherein the alkyl moiety is of 1-6 carbon atoms, or pyrrolidino;
m=1-4,q=1-3,and p=0-3;
any of the substituents R₁, R₂, R₃, or R₄ that are located on contiguous carbon atoms can together be the divalent radical —O—C(R8)2—O—;
or a pharmaceutically acceptable salt thereof with the proviso that when Y is —NH—, R₁, R₂, R₃, and R₄ are hydrogen, and n is 0, X is not 2-methylphenyl.

5. The method according to claim 4, wherein the EGFR kinase inhibitor is (4-dimethylamino-but-2-enoic acid [4-(3-chloro-4-fluoro-phenylamino)-3-cyano-7-ethoxy-quinolin-6-yl]-amide or a pharmaceutically acceptable salt thereof.

6. The method according to claim 1, wherein the cancer comprises colorectal and pancreatic cancer.

7. A method of treating or inhibiting cancer in a mammal in need thereof which comprises administering to said mammal an effective amount of capecitabine and (4-dimethylamino-but-2-enoic acid [4-(3-chloro-4-fluoro-phenylamino)-3-cyano-7-ethoxy-quinolin-6-yl]-amide.

8. A method of treating or inhibiting cancer in a mammal in need thereof which comprises administering to said mammal an effective amount of paclitaxel and (4-dimethylamino-but-2-enoic acid [4-(3-chloro-4-fluoro-phenylamino)-3-cyano-7-ethoxy-quinolin-6-yl]-amide.

9. A method of treating or inhibiting cancer in a mammal in need thereof which comprises administering to said mammal an effective amount of 5-FU and (4-dimethylamino-but-2-enoic acid [4-(3-chloro-4-fluoro-phenylamino)-3-cyano-7-ethoxy-quinolin-6-yl]-amide.

10. A method of treating or inhibiting cancer in a mammal in need thereof which comprises administering to said mammal an effective amount of cisplatin and (4-dimethylamino-but-2-enoic acid [4-(3-chloro-4-fluoro-phenylamino)-3-cyano-7-ethoxy-quinolin-6-yl]-amide.

11. A method of treating or inhibiting cancer in a mammal in need thereof which comprises administering to said mammal an effective amount of FOLFIRI and (4-dimethylamino-but-2-enoic acid [4-(3-chloro-4-fluoro-phenylamino)-3-cyano-7-ethoxy-quinolin-6-yl]-amide.

12. A method of treating or inhibiting cancer in a mammal in need thereof which comprises administering to said mammal an effective amount of FOLFOX4 and (4-dimethylamino-but-2-enoic acid [4-(3-chloro-4-fluoro-phenylamino)-3-cyano-7-ethoxy-quinolin-6-yl]-amide.
